(19) United States
(12) Patent Application Publication
(10) Pub. No.: US 2009/0142809 A1
(43) Pub. Date: Jun. 4, 2009

(54) Method for Cleaving Cervimycin Monoesters

(75) Inventors: Uwe Horn, Rottleben (DE); Armin Siering, Jena (DE); Gundela Peschel, Jena (DE); Christian Hertweck, Leipzig (DE); Peter-Jurgen Muller, Jena (DE); Bettina Bardl, Jena (DE); Jorg-Hermann Ozegowski, Jena (DE); Kerstin Herold, Jena (DE)

Correspondence Address: JORDAN AND HAMBURG LLP, 122 EAST 42ND STREET, SUITE 4000, NEW YORK, NY, 10168, US

(73) Assignees: LEIBNIZ-INSTITUT FÜR NATURSTOFF-FORSCHUNG UND INFE, Jena (DE); FRIEDRICH-SCHILLER-UNIVERSITÄT JENA, Jena (DE)

(21) Appl. No.: 12/084,721

(22) PCT Filed: Sep. 25, 2006
(86) PCT No.: PCT/DE2006/001709

§ 371 Date: May 29, 2008

(30) Foreign Application Priority Data
Nov. 8, 2005 (DE) 10 2005 053 670.0

(51) Int. Cl. C12P 19/56

(52) U.S. Cl. 435/78

(57) ABSTRACT

The invention relates to a method for cleaving cervimycin esters. An object of the invention to convert in a simple manner the cervimycin esters which are less active but formed in larger quantity into the highly active cervimycin K that occurs as a minor component in the preparation by fermentation, is achieved by preparing unesterified cervimycins from cervimycin esters with di- or monomethylated malonic acids, by ester cleavage effected by at least one esterolytic enzyme at temperatures of 20° C. to 75° C. and a pH of pH 5.0 to 10.0, preferably pH 6.0 to 9.0.

### BACKGROUND OF THE INVENTION

The invention relates to a method for cleaving cervimycin esters and generally serves to keep the people healthy by better combating infections thanks to the more effective production of an antibiotic.

During the last years, the frequency and spectrum of antimicrobial resistances considerably increased in Germany, and most of all in the European neighboring countries. Antibiotics that have been administered successfully so far lose their effectiveness against microbial pathogens. Therefore, novel antibiotics with significant activity against resistant pathogen microorganisms are intensively searched for. The feared interspecific transfer of vancomycin resistance from enterococci to staphylococci was observed in the USA in 2002 for the first time.

Cervimycins are an antibiotic group that is produced by Streptomyces sp. (archived as DSM 13059 at the DSZM, the “Deutschen Sammlung für Mikroorganismen und Zellkulturen” [German Collection of Microorganisms and Cell Cultures] in Braunschweig, Mascheroder Weg 1) during fermentation in a submerse culture (Herold K, Xu Z, Gollmick F A, Gräfe U, Hertweck C (2004); Biosynthesis of Cervimycin C, an aromatic polyketide antibiotic bearing an unusual dimethylmalonyl moiety. Org. Biomol. Chem. 7, 2411-2414 and Herold K (2005) Untersuchungen zur Struktur, Wirkungsweisen und Biosynthese der Cervimycine als Verbindungen einer besonderen Klasse aromatischer Polyketide [Investigations about the structure, effects and biosynthesis of cervimycins as compounds of a special class of aromatic polyketides.]. Thesis at the School of Biology and Pharmacy at the Friedrich Schiller University Jena. Date of defense: 17 Jan. 2005).

The cervimycins belong to the polyketide antibiotics; they consist of a polyketide aglycone to which one or two multicomponent saccharide chains are often linked.

It was found that a minor component of the cervimycins, cervimycin K, also known as HKI 10311129, exhibits an extremely strong activity against multiresistant staphylococci, even against the vancomycin-resistant Enterococcus faecalis.

Therefore, cervimycin K is a promising candidate for an effective antibiotic against resistant pathogens. It is a yellow-orange amorphous substance with the total molecular formula C₅₆H₇₅NaNO₂₂ and a molar mass of 1,136.4678 g/mol. Cervimycin K consists of a polyketide aglycone to which a disaccharide group and an unesterified tetrasaccharide group with terminal hydroxyl group are linked as sugar components. Unlike the majority of cervimycins, it does not contain a methylated malonic acid ester group. From 180 l of culture filtrate 13 mg were obtained.

For obtaining cervimycin K, the fermentation preparation can be extracted by using ethyl acetate, and then the extract is isolated from the liquid phase, dried with anhydrous sodium sulphate, and afterwards concentrated to dryness.

The oily residue is absorbed in a small amount of chloroform, the solution is filtered, and a crude product is precipitated by the addition of the 20fold volume of hexane.

Further processing is performed by gel permeation chromatography with Sephadex LH-20 by using methanol as an eluent. The antibacterially active center fraction is concentrated to dryness in vacuum. The fine purification is achieved by applying the silica gel chromatography method using a chloroform-methanol gradient.

The yellow fraction consisting of cervimycin K is concentrated to dryness.

Apart from the minor cervimycin K constituent, the culture solutions contain other, but less effective cervimycins in concentrations that range from 1.0 g/l to considerably higher values.

Among them are the cervimycins A and C that have already been protected as novel antibiotica named altamiramycin 2 and altamiramycin 1 (Groth I, Schlegel B, Kleinwächter P, Gräfe U, Härtl A, Perner A, Hilliger M, Möllmann U. New antibiotic altamiramycin, showing strong activity against Gram positive bacteria, obtained by culturing Streptomyces sp. HKI 0179 (DSM 13059). DE10065606 (2002-06-27)).

Altamiramycin 1 (cervimycin C) belonging to the main constituents has an acid amide function at the aglycone and said function is also present at cervimycin D and cervimycin K. Moreover, cervimycin C is esterified at the hydroxyl group of the tetrasaccharide with the dicarboxylic acid dimethyl malonic acid and cervimycin D with methyl malonic acid. It is a monoester of the dimethylated malonic acid. The cervimycin C methylester constituent is a diester of the dimethylated malonic acid; the second acid function is esterified with methanol. 1.6 g cervimycin C and 1.0 g cervimycin D were isolated from 180 l of culture filtrate (Herold K (2005)).

For their structural relatedness, the cervimycines C, D, produced in a relatively large amount, and the cervimycin C methylester should be used as initial substances for an efficient and profitable production of cervimycin K by cleaving the ester bond between the tetrasaccharide side-chain and the malonyl group. They all contain an acid amide group at the aglycone and only differ from cervimycin K by the presence of the methylated malonyl chain at the hydroxyl group of the tetrasaccharide side-chain.

It has been known for a long time that during the chemical hydrolysis of diesters of dicarboxylic acids, particularly of substituted malonic acid esters, in general one ester group is eliminated relatively easily by forming the monoester, whereas the second ester group is considerably more stable. The more complicated formation of the full esters of the malonic acids has possibly to do with the high negative charge of the free carboxyl group of the monoester produced in this process. The cleavage of an alcohol component of dicarboxylic acids by forming their monoesters is mostly performed under alkaline conditions. (H. G. O. Becker et al.: Organisch chemisches Grundpraktikum. [Basic organic-chemical practical course.] 7.1.4.3 Hydrolyse von Carbonsäurederivaten [Hydrolysis of carboxylic acid derivatives], publisher: Johann Ambrosius Barth Verlag Heidelberg, Leipzig, 20th edition 1996).

The latter also applies if the ester cleavage is performed by using enzymes that show an esterolytic activity, such as esterases, proteases, and lipases. Also here, the cleavage of the diester to a monoester is relatively easy, whereas the second ester group can often not be eliminated so that preferably the monoesters are formed.

As the reaction is stereospecific, enantiomer-enriched monoesters are preferentially formed from substituted prochiral monoalkyl malonic acid diesters. According to literature, the diesters are cleaved for example at pH 7 during 24 h with very high yields in some cases. Pork liver esterase (EC 3.1.1.1) is for example recommended to be used as the enzyme (Iriuchijima K, Hasegawa K, Tsuchihashi G (1982). Agric. Biol. Chem. 46, 1907; JP 04082863 A2; Heidel H, Huttner G, Vogel R, Helmchen G (1994) A novel chiral building block with neopentane framework for synthesis—chemoenzymatic preparation of R—CH₃C(CH₂OSO₂CF₃)(CH₂Cl)(CH₂Br) Chem. Ber. 127, 271-274).

The extent of the cleavage of mixed aryl and alkyl malonates with pork liver esterase has been increased by adding 50% or 25% dimethyl sulfoxide to the aqueous phase. If alpha chymotrypsin instead of pork liver esterase is used, very slow reactions or none were observed (Björkling F, Boutelje J, Gatenbeck K, Hult K, Norin T (1982) Tetrahedron Lett. 26, 4957 and Björkling F, Boutelje J, Gatenbeck K, Hult K, Norin T, Szmulik P (1985) Tetrahedron Lett, 41, 1347; Gutman A C, Shapiro M, Boltanski A (1992) Enzyme-catalyzed formation of chiral monosubstituted mixed diesters and half esters of malonic acid in organic solvents. J. Org. Chem. 57, 1063-1065).

The reactions can also be performed in aqueous phosphate-buffered solutions at pH 8 (Schneider M, Engel N, Boensmann H (1984) Angew. Chemie [Applied chemistry] Int. Ed. Engl. 23, 66). The addition of methanol (10% v/v) increased the yield but the stereoselectivity of the reaction became worse. In the presence of dimethylsulfoxide, the reaction rate decreased (Luyten M, Müller S. Herzog B, Keese R (1987) Helv. Chim. Acta 70, 1250).

Tests to cleave the malonic acid groups of cervimycin C, the cervimycin C methylester or cervimycin D by chemical hydrolysis in a neutral and alkaline or acid pH range to gain cervimycin K has not been successful yet. Under extreme pH conditions considerably smaller cleavage products of the cervimycins were gained. Most likely, the basic structure of the cervimycin molecules is destroyed in the hydrolysis under alkane or acid pH conditions.

All the enzymatic transformations at substituted malonic acid esters described in literature have been performed by using malonic acid esters that have a very simple structure. Without exception, the monoesters of the used malonyl acid derivatives were always produced in these reactions.

The production of the substituted malonic acids by cleaving the two ester bonds and the yield of the alcohol components have not been described yet, because normally the substituted malonic acids and the alcohol components are directly synthesized. Like the transformation of cervimycin C (identical to altamiramycin 1), cervimycin D, and cervimycin C-methyl ester to cervimycin K by using esterolytic enzymes for catalysis, the extraction of complex alcohol constituents from natural material by cleaving their malonyl monoesters has not been described up to now.

As it is the case for all ester cleavages, a thermodynamic equilibrium is established in the reaction liquids, but this development can possibly take an extremely long time. By the addition of enzymes that have a catalytic effect on the corresponding ester bonds, the velocity of equilibrium establishment can be increased in favorable cases.

As a rule, esters that are formed rapidly are also cleaved rapidly. But according to this rule, such high-complex esters like the cervimycin ones should be kinetically very stable and cannot be enzymatically cleaved at all or only very slowly. This rule applies even more for the second ester bond at the methylated malonic acids.

### SUMMARY OF THE INVENTION

It is the object of the present invention to convert, in a simple manner, the cervimycin esters, which are less active but formed in larger quantity, to the highly active cervimycin K that appears as a minor component in the production by fermentation.

Other objects and advantages of the invention will be apparent from the following description of the invention.

The invention is based on the treatment of structurally suitable main components, such as cervimycin dimethyl malonic acid monoesters and methyl malonic acid monoesters and the cervimycin dimethyl malonic acid diesters, as educts in the course of a reaction with esterolytically active enzymes at temperatures ranging between 20° C. and 75° C., preferably between 30 and 60° C., at a pH from pH 5.0 to 10.0, preferably from pH 6.5 to 9.0, in a reactor for a period from 0.1 hour to several weeks, preferably 0.1 hour to 48 hours.

Surprisingly it was found that the reaction takes place regardless whether an acid amide group exists at the aglycone as it is the case for cervimycin C (altamiramycin I), cervimycin D, cervimycin J or cervimycin C-methyl ester or whether the amino group of the acid amide group is substituted by a methyl group, as it is the case for cervimycin A (altamiramycin 2), cervimycin B, cervimycin L or the cervimycin A methyl ester.

Furthermore, it was surprisingly found that the formation of cervimycin K from the cervimycin monoesters will be considerably increased or a cleavage reaction can only be proven if a non-enzymatic active protein, such as serum albumin, exists in concentrations that range from 0.1% through 5%, preferably from 0.5% to 2%, in the reaction preparation.

Esterases in the narrower sense, but also proteases and lipases are used as esterolytic enzymes. So, lipases from fungi of the genuses mucor, rhizomucor, rhizopus, candida, aspergillus, from Humicola lanuginosa or from Pseudomonas fluoreszence are successfully used for this purpose. The use of pork liver esterase or of proteases of microbial origin, such as pronase from streptomyces griseus, cobalt metalloprotease MO2 from Streptomyces hygroscopicus (DD 263301), subtilisin or alcalase and also esterases which are produced from the cervimycin creator streptomyces sp. (DSM 13059) and of Streptomyces tendae as well as proteases which—like papin—are obtained from plants, are also within the scope of the present invention.

It was also surprisingly found that the addition of surfactants increases the conversion of the educts. So, in a test in which a lipase from Rhizopus niveus has been used, the addition of Triton X100 caused an increase in conversion from 10% for a non-supplemented preparation to 20%.

The inventive reaction takes place in a homogeneous phase as well as in a heterogeneous phase with one phase being a liquid one. In one inventive embodiment the liquid milieu consists of an aqueous, buffered reaction solution having a conductivity less than 10 mS. The solution can additionally contain hydrosoluble solvents, such as methanol or other lower alcohols or dimethyl formamide or dimethyl sulfoxide. By adding these solvents the solubility of the cervimycin educts is increased so that they can be used in higher concentration in the reaction.

The conversion rate can also be increased by using a solvent which cannot be mixed with water, as a second liquid phase. The second, nonaqueous phase can be, for example, hexane or octane.

The invention allows the reaction in the presence of a solid phase, too. In one embodiment, ion exchangers, preferably Q Sepharose or DEAE-cellulose is used for this purpose. The advantage of this method is that intensive pH changes are avoided. It may be assumed that the methylated malonic acids released in this reaction remain at the exchanger and thus they are eliminated from the equilibrium. By using an alkaline-set anion exchanger the released proteins can be further neutralized so that the pH changes are avoided.

In a further method, the methyl malonic acid esters or the dimethyl malonic acid monoesters of the cervimycins, which are covalently bonded to a solid phase via a spacer containing 3 to 16 carbon atoms in the chain, are used for the reaction. Preferably, such solid phases are used the surfaces of which carry hydroxyl groups and are coated with aminopropyltriethoxysilane and to which the cervimycin monoesters are covalently bonded according to the carbodiimide method.

In another embodiment, the relatively small malonic acids monomethylated with a molar mass of 119.1 g/mol or dimethylated with 134.1 g/mol are continuously removed from the reactor volume during the reaction by ultra-filtration through membranes or other penetration barriers with a cut-off equal or smaller than 1 kD, whereas the esterolytic enzyme cannot pass them. The molar masses of the enzymes are normally between 30 and 60 kD. The procedure is performed in a generally known ultra-filter system. Membrane modules or hollow fiber modules are used as penetration barriers. Repumping and application of a pressure of up to 2 atm cause a permeate flow through the penetration barrier, and said flow mainly carries or dilutes smaller reaction products. The pressure selected must have such a value that the cervimycin educts permeate only to a small extent.

Another method uses the penetration barriers, preferably arranged as membrane modules or hollow fibers, to quasi immobilize the enzyme. In dependence of the selection behavior, the cut-off of the barrier ranges between 1 kD and 30 kD in this method. In this embodiment, the educts and also the reaction products permeate relatively rapidly through the penetration barrier to the enzyme and also backwards.

In another embodiment, the esters of cervimycins C and D are bonded to anion exchangers before or also during the mixture of the reactants and the esterases in dissolved condition act on the cervimycins C and D that are bonded to the malonic acid carboxyl group at the ion exchanger. Also in this method, Q-Sepharose or DEAE-Sepharose is preferably used as an anion exchanger. By mixing the anion exchanger with the monoesters cervimycin C and cervimycin D ion exchangers/cervimycin complexes are produced in which the cervimycin esters are cleaved very probably in a higher rate in a steric condition that is favorable for the attack of the esterolytic enzymes. After the cleavage the produced methylated malonic acid remains at the exchanger.

In one embodiment, the formation of the complexes can be performed in a separate step before the ester cleavage. The disadvantage of this method is the fact that the methyl ester of cervimycin C is not bonded here and therefore it does not take part in the reaction.

In another embodiment, anion exchangers and cervimycin esters are already mixed in the reactor and the enzyme is added afterwards. As the methyl ester of the cervimycin as a monoester is cleaved quickly and quantitatively to cervimycin C also without being bonded to ion exchangers, losses are not caused in this method.

Furthermore, it was found that with the use of ion exchangers also in smaller quantities than required for the formation of a 1:1 complex with the cervimycin esters, the desired ester cleavage takes place after adding the esterolytic enzyme.

The produced cervimycin K changes to the liquid phase in all inventive embodiments, whereas the produced methyl and dimethyl malonic acids remain at the exchanger.

Surprisingly, it was also found that the bondage of the esterolytic enzyme to solid phases shifts the reaction equilibrium in favor of the reaction products. In this embodiment, the educts and the reactants are led past the immobilized enzyme and the produced malonic acids are removed from the reaction system by their bondage to anion exchangers with suitable, normally low ionic strengths. Adsorbents, such as porous glass, and also supramolecular compounds, like zeolites and/or dextrans, are used as solid carriers.

Moreover, the supramolecular compounds have very probably a positive effect on the equilibrium condition because they catch the produced methylized malonic acids via their nanoscaled pores or they have a positive steric effect on the reaction.

The use of cross-linked or immobilized esterolytic enzymes does not only influence the equilibrium condition or the velocity of the reaction in a positive manner, but it has also the advantage that said enzymes can be recovered or used several times.

The inventive method is also characterized by the facts that the reaction equilibrium or the rate of reaction can be shifted in favor of the reactants by adding bivalent or trivalent cations, for example Mg⁺⁺, Mn⁺⁺, Zn⁺⁺, Fe⁺⁺⁺ or Al⁺⁺⁺ in concentrations over 10 mM, and that in this way the formation of cervimycin K is supported. The formation of sparingly soluble salts of the released malonic acids is very probable and thus said acids are removed from the equilibrium system.

The inventive method also extends to the fermentation with streptomyces sp. (DSM 13059) or another microorganism of the streptomyces tendae type forming the cervimycins. By the addition of esterolytic enzymes, preferably in a late phase of the fermentation, a conversion of the cervimycin esters to cervimycin K is already achieved during the fermentation process in this manner.

In another embodiment, the cell-free culture filtrate of the fermentation is treated with esterolytic enzymes. To preclude the negative influence of living microorganisms on the reaction and to get also a fouling-free reaction preparation, which is stable over a long, time, the reaction solutions are sterilized by filtering them in sterile filters.

### DETAILED DESCRIPTION OF THE INVENTION

### Example 1

40 μl of a methanolic cervimycin C solution, which contains 2 mg of cervimycin C in 0.5 ml methanol, is added to 1 ml of a 0.1 M phosphate buffer at pH 8.5. 40 μl of said solution are added to 1 ml phosphate buffer at pH 8.5. 40 μl of a lipase solution are added to the mixture. Said lipase solution contains 2 mg of the lipases that are indicated below and has a specific activity of 2.6 U/g (Fluka) in 0.5 ml water. By the addition of 0.1 M NaOH the preparation is incubated at 40° C. over a period of 50 h and during this time the pH-value is controlled and kept at a constant level. By using the HPLC method, the increase of the cervimycin K content from 0 mg/l to 6.9 mg/l in the mixture is proven whereas the concentration of cervimycin C decreases from 40 mg/l to approximately 34 mg/l.

In the aqueous phase, the concentration of the cervimycins is determined by a standardized HPLC method.

HPLC parameters for determining the cervimycins

HPLC: low pressure gradient system of the Jasco company

Detector: diode array

Column: ProntoSIL 120-5-C18-ace-EPS 5 μm, 250×4 mm

Pre-column cartridge: ProntoSIL 120-5-C18-ace-EPS 5 μm 20×4 mm

Column temperature: 25° C.

Mobile phase: ACN: TFA (0.1%) gradient

0 min-50% ACN: 50% TFA

20 min-100% ACN

30 min-100% ACN

30.5 min-50% ACN: 50% TFA

35 min-50% ACN: 50% TFA

Flow rate: 1 ml/min

Injection volume: 20 μl

### Example 2

250 μl of a methanolic cervimycin C solution, which contains 20 mg cervimycin C in 1 ml methanol, are added to 5 ml of a 0.01 M phosphate buffer at pH 7.03 that contained 0.5% of bovine serum albumin in a further test procedure. Afterwards, 0.5 ml of a 0.01 M phosphate-buffered esterase solution (esterase from horse liver), pH 7.03, which contains 10 mg of esterase per ml of 0.01 M phosphate buffer at pH 7.03, are added by mixing. The mixture is incubated at 37° C. for 24 h.

To prove the cervimycin K, 0.1 ml are taken from the mixture and cleaved by HPLC (Shimadzu) with an acetonitrile—0.1% trifluoro-acetic acid gradient on a ProntoSIL ace-EPS 120-5-C₁₈ column (250×4 mm). The cervimycin K peak is analyzed in a mass spectroscopy method (Bentrop mass spectrometer Finnigan L C Q, Finnigan, Bremen) in connection with an electron spray ion source and an ion-trap analyst. A characteristic mass peak is proven for cervimycin K for M 1113. The quantitative analysis shows a cervimycin K content of 114 mg/l in the sample. This quantity corresponds to a yield of 18%. (Refer to Table 1.)

TABLE 1

| | Cervimycin derivatives produced after 24 h | | | | |
|---|---|---|---|---|---|
| | Cervimycin | Cervimycin | Cervimycin | Cervimycin | Cervimycin |
| Test conditions | K (mg/ml) | D | C | B | A |
| 5 ml buffer, without enzyme, | 1.49 | 27.2 | 657.6 | 0 | 1.86 |
| 0.25 ml cervimycin C solution | | | | | |
| 5 ml buffer, 0.5 ml enzyme, | 0.14 | 1.05 | 580.0 | 0 | 0.10 |
| 0.25 ml cervimycin C solution | | | | | |
| 5 ml buffer, 0.5% bovine | 114.3 | 9.42 | 278.6 | 0 | 0.08 |
| serum albumin, 0.5 ml | | | | | |
| enzyme, 0.25 ml cervimycin C | | | | | |
| solution | | | | | |

### Example 3

Cleavage of cervimycin C by using different esterases and lipases:

2 mg of an enzyme dried by lyophil are dissolved in 0.02 M phosphate buffer at pH 8.5 (enzyme solution). 40 μl of the methanolic cervimycin solution and 20 μl of the enzyme solution are given to 1 ml phosphate buffer in the preparation. The cervimycin K concentration indicated in the right column is produced from 40 mg/L cervimycin.

(Refer to Table 2.)

TABLE 2

| | Activity delivered | | Cervimycin |
|---|---|---|---|
| Enzymes | by the supplier | | K |
| Lipase Rizopus arrhizus | 2 | U/g | 2.46 mg/l |
| Lipase Candida cylindrecea | 2.3 | U/mg | 2.76 mg/l |
| Lipase Pseudemonas cepacia | 48 | U/mg | 2.94 mg/l |
| Lipase Aspergillus niger | 1 | U/mg | 1.31 mg/l |
| Lipase Rhizopus niveus | 2.6 | U/g | 6.87 mg/l |
| Lipoprotein Lipase dog pancreas | 23.3 | U/mg | 3.64 mg/l |
| Lipase Pseudomanoas fluoreszenz | 42.4 | U/mg | 1.83 mg/l |
| Lipase Mucor mihei | 1.3 | U/mg | 4.48 mg/l |
| Lipase Rhizomucor mihei | 0.51 | U/mg | 1.03 mg/l |
| Lipase of wheatgerms | 0.08 | U/mg | 0.04 mg/l |
| Esterase Streptomyces | 50 | U/mg | 0.14 mg/l |
| diastatochromogenes | | | |
| Pork liver esterase | 34 | U/mg | 0.28 mg/l |

### Example 4

1.0 ml of a methanolic solution, which contains 640 μg/ml cervimycin C and 1 g Q-Sepharose equilibrated with 0.005 M phosphate buffer, are added to 10 ml of a 0.005 M phosphate buffer at pH 8.5. After the bonding of the cervimycin C to the ion exchanger, the clear supernatant is removed by centrifugation and 5 ml of a lipase solution, which contains 2 mg lipase from Rhizomucor mihei with a specific activity of 0.51 U/mg (Fluka), are added to the ion exchanger. The suspension is incubated whole mixing it at 40° C. over a period of 50 h. About 15% cervimycin C are converted to cervimycin K. The produced cervimycin K is contained in the aqueous supernatant.

For the preparation, the suspension is extracted by the extraction with ethyl acetate, and then the extract is separated from the aqueous phase, dried with anhydrous sodium sulfate, and afterwards concentrated to dryness. The oily residue is resorbed in a little chloroform, the solution is filtered and a crude product is precipitated by the addition of a 20fold volume of hexane. The further processing is made by gel permeation chromatography at Sephadex LH-20 by using methanol as eluent. The center antibacterial fraction is concentrated to dryness in vacuum. The fine purification is achieved by applying the silica gel chromatography method using a chloroform-methanol gradient. The yellow fraction consisting of cervimycin K is concentrated to dryness.

1.-42. (canceled)

43. Method for producing unesterified cervimycin, comprising reacting in a reaction medium a di- or monomethylated malonic acid ester of cervimycin with at least one estrolytic enzyme comprising an esterase or a lipase, at temperatures from 20° C. to 75° C. and a pH of 5.0 to 10.0, for a period of 0.1 hour to 48 hours in presence of a non-enzymatic active protein in a concentration of 0.1% to 5.0%.

44. Method as set forth in claim 43, wherein the non-enzymatic protein comprises albumin.

45. Method as set forth in claim 44, wherein the albumin comprises a cow albumin.

46. Method as set forth in claim 43, wherein the cervimycin malonic acid ester comprises cervimycin C and/or cervimycin D and/or the methyl ester of cervimycin C.

47. Method for producing an unesterified cervimycin, comprising reacting in a reaction medium a di- or monomethylated malonic acid ester of cervimycin with at least one esterase comprising an esterase from a liver of a mammal, at temperatures from 20° C. to 75° C. and a pH of 5.0 to 10.0 for a period of 0.1 hour to 48 hours.

48. Method as set forth in claim 47, wherein the reaction is conducted in presence of a non-enzymatic active protein in a concentration of 0.1% to 5%.

49. Method according to claim 47, wherein the esterase comprises a pork liver esterase or a horse liver esterase.

50. Method for producing unesterified cervimycin, comprising reacting in a reaction medium a di- or monomethylated malonic acid ester of cervimycin with at least one lipase comprising a lipase from a pseudomonas or a streptomyces, at temperatures of 20° C. to 75° C., and a pH of 5.0 to 10.0 for a period of 0.1 hour to 48 hours.

51. Method as set forth in claim 50, wherein the reaction is conducted in presence of a non-enzymatic active protein in a concentration of between 0.1% and 5%.

52. Method according to claim 50, wherein the lipase comprises a lipase from Pseudomonas fluoreszenz, Streptomyces tendae or Streptomyces sp. (DSM 13059).

53. Method for producing unesterified cervimycin, comprising reacting in a reaction medium a di- or monomethylated malonic acid ester of cervimycin with at least one lipase comprising a lipase from rhizomocur, at temperatures of 20° C. to 75° C. and a pH of 5.0 to 10.0 for a period of 0.1 hour to 48 hours.

54. Method as set forth in claim 53, wherein the reaction is conducted in presence of a non-enzymatic active protein in a concentration of 0.1% to 5%.

55. Method according to claim 53, wherein the lipase comprises a lipase from Rhizomucor mihei or Rhizomucor javanicus.

56. Method for producing unesterified cervimycin, comprising reacting in a reaction medium a di- or monomethylated malonic acid ester of cervimycin with at least one lipase comprising a lipase from Mucor, at temperatures of 20° C. to 75° C. and a pH of 5.0 to 10.0 for a period of 0.1 hour to 48 hours.

57. Method as set forth in claim 56, wherein the reaction is conducted in presence of a non-enzymatic active protein in a concentration of 0.1% to 5%.

58. Method according to claim 56, wherein the lipase comprises a lipase from Mucor mihei.

59. Method for producing unesterified cervimycin, comprising reacting in a reaction medium a di- or monomethylated malonic acid ester of cervimycin with at least one lipase comprising a lipase from rhizopus, at temperatures of 20° C. to 75° C. and a pH of 5.0 to 10.0 for a period of 0.1 hour to 48 hours.

60. Method as set forth in claim 59, wherein the reaction is conducted in presence of a non-enzymatic active protein in a concentration of 0.1% to 5%.

61. Method according to claim 59, wherein the lipase comprises a lipase from Rhizopus niveus, Rhizopus arrhizus, Rhizopus japanicus, Rhizopus delemar or Rhizopus rhizopodiformis.

62. Method for producing and unesterified cervimycin, comprising reacting in a reaction medium a di- or monomethylated malonic acid ester of cervimycin with at least one lipase comprising a lipase from candida, at temperatures of 20° C. to 75° C. and a pH of 5.0 to 10.0 for a period of 0.1 hour to 48 hours.

63. Method as set forth in claim 62, wherein the reaction is conducted in presence of a non-enzymatic active protein in a concentration of between 0.1% and 5%.

64. Method according to claim 62, wherein the lipase comprises a lipase from Candida cylindracea.

65. Method for producing unesterified cervimycin, comprising reacting in a reaction medium a di- or monomethylated malonic acid ester of cervimycin with at least one lipase comprising a lipase from aspergillus, at temperatures of 20° C. to 75° C. and a pH of 5.0 to 10.0 for a period of between 0.1 hour to 48 hours.

66. Method as set forth in claim 65, wherein the reaction is conducted in presence of a non-enzymatic active protein in a concentration of 0.1% to 5%.

67. Method according to claim 65, wherein the lipase comprises a lipase from Aspergillus niger.

68. Method for producing unesterified cervimycin, comprising reacting in a reaction medium a di- or monomethylated malonic acid ester of cervimycin with at least one lipase comprising a lipase from Humicola lanuginosa, at temperatures of 20° C. to 75° C. and a pH of 5.0 to 10.0 for a period of 0.1 hour to 48 hours.

69. Method according to claim 68, wherein the reaction is conducted in presence of a non-enzymatic active protein in a concentration of between 0.1% and 5%.

70. Method as set forth in claim 43, wherein the reaction medium also contains at least one protease or proteinase comprising α-chymotrypsin, subtilisin, alcalase, cobalt-dependent metalloprotease MO2 from Streptomyces hygroscopicus, pronase or papain.

71. Method as set forth in claim 43, wherein the esterolytic enzyme is in an immobilized form.

72. Method as set forth in claim 43, wherein the esterolytic enzyme is bonded to a solid.

73. Method according to claim 72, wherein the solid comprises an anion exchanger comprising Q-Sepharose or DEAE-Sepharose.

74. Method as set forth in claim 72 or 73, wherein the bonded estrolytic enzyme is suspended in the reaction medium.

75. Method as set forth in claim 72 or 73, wherein the reaction medium comprises water and a hydrosoluble organic solvent, or water and hexane or octane.

76. Method as set forth in claim 43, wherein the esterolytic enzyme is chemically cross-linked.

77. Method as set forth in claim 43, wherein the cervimycin malonic acid ester is bonded to an anion exchanger.

78. Method as set forth in claim 43, wherein the cervimycin malonic acid ester is covalently bonded to a solid via a spacer comprising a chain of 3 to 16 carbon atoms.

79. Method as set forth in claim 43, further comprising separating the malonic acid ester resulting from the reaction from the enzyme by means of an ultra-filtration apparatus having a cut-off in the range from 0.1 kD to 1 kD or a cut-off in a range of 1.0 kD to 30 kD.

80. Method as set forth in claim 72, wherein the solid comprises a compound which adsorbs proteins.

81. Method as set forth in claim 72, wherein the solid comprises a nano-structured porous glass or a supramolecular compound comprising a zeolite or a dextran.

82. Method as set forth in claim 72, wherein the reaction is conducted in a reaction preparation further comprising at least one bivalent or trivalent cation of a metal in a total concentration higher than 10 mM and at least one surfactant and the pH value is maintained at a constant level.

83. Method as set forth in claim 43, wherein the reaction medium comprises a culture fluid for a fermentation that is performed with Streptomyces sp. (DSM 13059) or with a cervimycin-forming strain of Streptomyces tendae whereby the di- or monomethylated malonic acid ester of cervimycin is contained in the culture fluid and the at least one estrolytic enzyme is added to the culture medium during or at an end phase of the fermentation.

84. Method as set forth in claim 43, wherein the reaction medium comprises a cell-free, sterilely filtered culture fluid that contains the di- or monomethylated malonic acid ester of cervimycin and the at least one estrolytic enzyme is added to the culture medium.
